(19) United States
(12) Patent Application Publication
(10) Pub. No.: US 2024/0342140 A1
(43) Pub. Date: Oct. 17, 2024

(54) TOPICAL LIQUID COMPOSITIONS FOR TREATING HAIR LOSS

(71) Applicant: Chemistry RX, FOLCROFT, PA (US)

(72) Inventor: Lars BRICHTA, Brooklyn, NY (US)

(21) Appl. No.: 18/755,647

(22) Filed: Jun. 26, 2024

Related U.S. Application Data
(63) Continuation-in-part of application No. 17/302,777, filed on May 12, 2021.
(60) Provisional application No. 63/023,279, filed on May 12, 2020.

(51) Int. Cl. A61K 31/436; A61K 9/00; A61K 9/08; A61K 31/506; A61K 31/519; A61K 31/573; A61K 47/10; A61K 47/12; A61K 47/20

(52) U.S. Cl. CPC A61K 31/436; A61K 9/0014; A61K 9/08; A61K 31/506; A61K 31/519; A61K 31/573; A61K 47/10; A61K 47/12; A61K 47/20

(57) ABSTRACT

Disclosed herein are liquid compositions and methods for treating hair loss by topical administration of the liquid compositions to a subject in need thereof.

### CROSS-REFERENCE TO RELATED APPLICATIONS

This application is a continuation-in-part of U.S. patent application Ser. No. 17/302,777, filed on May 12, 2021, which claims priority from U.S. Provisional No. 63/023,279, entitled “Compositions for Treating Hair Loss,” filed May 12, 2020, the entirety of which is hereby incorporated by reference in its entirety.

### SUMMARY OF THE INVENTION

Various embodiments of the invention are directed to methods for treating hair loss comprising topically administering to a patient in need of treatment a liquid composition containing an active agent selected from the group consisting of minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and a solvent. The patient in such methods may have a disorder such as alopecia areata, alopecia totalis, alopecia universalis, vitiligo, graft versus host disease, telogen effluvium, tinca capitis (dermatophytosis), hypotrichosis, hereditary hypotrichosis simplex, frontal fibrosing alopecia, cicatricial alopecia, lichen planopilaris, ring alopecia, and chemotherapy induced alopecia. In some embodiments, the active agent may be tacrolimus. In various embodiments, the active agent may have a concentration of about 0.1% (w/w) to about 15% (w/w) based on the total composition. In some embodiments, the solvent may be phosphate buffered saline solution, water, or saline, and in some embodiments, the solvent may be water, isopropyl alcohol, dipropylene glycol methyl-ether, butylated hydroxytoluene dipropylene glycol monomethyl-ether, 1-methoxy 2-propanol (glysolv PM/lcinol PM), ethylene glycol monobutylether (butyl glyxolv/butyl icinol), butyl di glysolv (butyl-icinol), transcutol, propylene glycol (PG), N-methyl-2 pyrrolidone (NMP), methylene chloride, diethyl ether, ethanol, dimethyl sulfoxide (DMSO), lactic acid, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, plant oils, Aloe vera derivatives or sesame seed oil or derivatives thereof, ethosomes, azone, castor oil derivatives, ethoxylated castor oil, jojoba oil derivatives, corn oil derivatives, emu oil derivatives, and combinations thereof.

In certain embodiments, the solvent may be an oil in an aqueous solution. The oil in such embodiments may be coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof. In some embodiments, the oil may have a concentration of about 0.5% (w/w) to about 10% (w/w) based on the total weight of the composition. The aqueous phase may be water or a polar water-miscible solvent such as, for example, Cl-C4 alcohols, polyethylene glycol, ethylene glycol, propylene glycol, butylene glycol, pentylene glycol, glycerol, diethylene glycol monocthyl ether, propylene carbonate, and combinations thereof. In some embodiments, the polar water-miscible solvent may have a concentration of about 0.5% (w/w) to about 10% (w/w) based on the total composition.

In some embodiments, the composition may further include about 0.1 wt % to about 5 wt % surfactant based on the total weight of the composition. The surfactant may be alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters, Span 20 or Span 80, block copolymers of ethylene oxides and propylene oxides, Pluronic Ll21 or Pluronic F68, polymeric surfactants having crosslinked copolymers of acrylic acid, Pemulen Tr-I and Pemulen Tr-2, and combinations thereof in such embodiments. In some embodiments, the composition may further include about 1% (w/w) to about 20% (w/w) penetration enhancer based on the total weight of the composition. In some embodiments, the composition may include an antioxidant such as, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, crythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

Further embodiments are directed to compositions containing an active agent such as minoxidil, finasteride, dutasteride, tofacitinib, tacrolimus, bimatoprost, latanoprost, spironolactone, aldactone, kenalog-10, kenalog-40, triamcinolone, azulfidine, sulfasalazine, sulfazine, and the like and combinations thereof and an oil in an aqueous solution. In some embodiments, the compositions are liquid compositions, or solutions, containing the active agent, at least one solvent, and a carrier.

The oil in such embodiments may be coconut oil, squalane, isopropyl myristate, isopropyl isostearate, isopropyl palmitate, modified triglycerides, caprylic capric glycerides, fractionated triglycerides, glyceryl tricaprate, glyceryl tricaprylate, glyceryl tricaprylate, glyceryl tricaprylate/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8- C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and mixtures thereof. In some embodiments, the oil may have a concentration of about 0.5% (w/w) to about 10% (w/w) based on the total weight of the composition. The aqueous phase may be water or a polar water-miscible solvent such as, for example, CI-C4 alcohols, polyethylene glycol, ethylene glycol, propylene glycol, butylene glycol, pentylene glycol, glycerol, diethylene glycol monoethyl ether, propylene carbonate, and combinations thereof. In some embodiments, the polar water-miscible solvent may have a concentration of about 0.5% (w/w) to about 10% (w/w) based on the total composition.

In some embodiments, the composition may further include about 0.1 wt % to about 5 wt % surfactant based on the total weight of the composition. The surfactant may be alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters, Span 20 or Span 80, block copolymers of ethylene oxides and propylene oxides, Pluronic Ll21 or Pluronic F68, polymeric surfactants having crosslinked copolymers of acrylic acid, Pemulen Tr-I and Pemulen Tr-2, and combinations thereof in such embodiments. In some embodiments, the composition may further include about 1% (w/w) to about 20% (w/w) penetration enhancer based on the total weight of the composition. In some embodiments, the composition may include an antioxidant such as, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, crythorbic acid, gum guaiac, propyl gallate, thiodipropionic acid, dilauryl thiodipropionate, tert-butylhydroquinone, tocopherol, and pharmaceutically acceptable salt and ester thereof, and combinations thereof.

### DETAILED DESCRIPTION:

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

All percentages, parts and ratios are based upon the total weight of the topical compositions and all measurements made are at about 25° C., unless otherwise specified.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g. “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum comeum or stratum spinosum. For example, 1,3-propanediol is a carrier.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopcia for use in mammals (e.g. animals), and more particularly, in humans.

The term “appreciable” is employed herein to refer to those agents of interest/compounds, salts, compositions, dosage forms, etc., which-within the scope of sound medical judgement-resulted in increased hair growth. An improvement in hair growth may be quantified by a SALT score or by a % regrowth measurement. A positive appreciable change in hair growth may not rise to a pharmaceutically acceptable or cosmetically acceptable determination.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

The term “SALT” refers to the Severity of Alopecia Tool, which is a statistical measurement that may be used by those skilled in the art to quantize change in the severity of alopecia in a patient or a sample of patients overall. A negative change in SALT score indicates an improvement in a subject's condition.

As used herein, the term “pharmaceutically acceptable carrier” refers to any of the standard pharmaceutical carriers including, but not limited to non-toxic solvent, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to liquid compositions containing one or more active agents such as, for example, minoxidil, finasteride, dutasteride, tofacitinib, tacrolimus, bimatoprost, latanoprost, spironolactone, aldactone, kenalog-10, kenalog-40, steroids, such as clobetasol propionate and triamcinolone, azulfidine, sulfasalazine, sulfazine, and the like, and combinations thereof. Such active agents can be provided in any amount capable of providing treatment. For example, the compositions of embodiments may include up to about 15% (w/w), about 0.25% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), about 0.1% (w/w) to about 0.5% (w/w) or any range or individual concentration of active agent encompassed by these example ranges. Such liquid compositions may be administered to the scalp of a subject experiencing hair loss without the matting, weighing down, and oiliness associated with creams and ointments currently used to deliver active agents to the scalp.

Example compositions may include various known components. For example, in some embodiments, the composition may include a solvent such as water, isopropyl alcohol, dipropylene glycol methyl-ether, butylated hydroxytoluene dipropylene glycol monomethyl-ether, 1-methoxy 2-propanol (glysolv PM/lcinol PM), ethylene glycol monobutylether (butyl glyxolv/butyl icinol), butyl di glysolv (butyl-icinol), transcutol, propylene glycol (PG), N-methyl-2 pyrrolidone (NMP), methylene chloride, diethyl ether, ethanol or ethyl alcohol, dimethyl sulfoxide (DMSO), lactic acid, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, ethosomes, azone, castor oil derivatives, such as ethoxylated castor oil, jojoba oil derivatives, corn oil derivatives, emu oil derivatives, and the like and combinations thereof. The solvent can be present in a concentration of about 20% (w/w) to about 99.9% (w/w), about 60% (w/w) to about 99.8% (w/w), about 75% (w/w) to about 99.5% (w/w), about 80% (w/w) to about 100% (w/w), or any range or individual concentration of solvent encompassed by these example ranges.

In some embodiments, the solvent may be a mixture of solvents. For example, the composition may contain an oil in an aqueous solution. The one or more of the one or more active agents may be dissolved in the oil phase. Examples of oils that can be used in the compositions include coconut oil, squalane, isopropyl myristate, isopropyl isostearate, isopropyl palmitate, modified triglycerides, caprylic capric glycerides, fractionated triglycerides, glyceryl tricaprate, glyceryl tricaproate, glyceryl tricaprylate, glyceryl tricaprylate/caprate, glyceryl tricaprylate/caprate, glyceryl tricaprylate/caprate/laurate, glyceryl tricaprylate/caprate/linol cate, glyceryl tricaprylate/caprate/stearate, glyceryl trilaurate, glyceryl trilinoleate, glyceryl trilinolenate, glyceryl trioleate, glyceryl triundecanoate, linoleic glycerides, saturated polyglycolized glycerides, synthetic medium chain triglyceride containing primarily C8-C12 fatty acid chains, medium chain triglycerides, long chain triglycerides, modified triglycerides, fractionated triglycerides, isostearyl isostearate, diisopropyl adipate, mineral oil, dimethicone, cyclomethicone, hydrogenated polyisobutene, heptamethylnonane, and the like and mixtures thereof. In various embodiments, the compositions may include an oil at a concentration of about 0.5% (w/w) to about 10% (w/w), about 1% (w/w) to about 5% (w/w), about 0.5% (w/w) to about 5% (w/w), or any range or individual concentration of solvent encompassed by these example ranges.

In some embodiments, the aqueous phase may be water. In other embodiments, the aqueous phase may include a polar water-miscible solvent, such as an alcohol or glycol. Polar water-miscible solvents may improve skin penetration and salvation of the active agent. The polar water-miscible solvent may be, for example, C₁-C₄ alcohols, polyethylene glycol, ethylene glycol, propylene glycol, butylene glycol, pentylene glycol, glycerol, diethylene glycol mono ethyl ether, propylene carbonate, and the like and combinations and mixtures thereof. The total amount of polar water-miscible solvent may be less than about 10 wt % by weight of the total composition, about 0.5% (w/w) to about 10% (w/w), about 1% (w/w) to about 5% (w/w), about 0.5% (w/w) to about 5% (w/w), or any range or individual concentration of solvent encompassed by these example ranges.

In some embodiments, the compositions may include a surfactant. The surfactant may be incorporated into the oil phases, the aqueous phase, or both. Suitable surfactants include, for example, alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters (such as Span 20 or Span 80), block copolymers of ethylene oxides and propylene oxides (such as Pluronic Ll21 or Pluronic F68), polymeric surfactants having crosslinked copolymers of acrylic acid, such as Pemulen Tr-I and Pemulen Tr-2, and the like and combinations and mixtures thereof. The composition may include surfactant in a concentration of about 0.1 wt % to about 5 wt %, about 0.5 wt % to about 3 wt %, about 0.7 to about 2 wt %, or any range or individual concentration of surfactant encompassed by these example ranges.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, crythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include a UV-absorbing compound such as, for example, glyceryl PABA, padimate O, roxadimate, dioxybenzone, oxybenzone, sulisonbenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of UV-absorbing compound may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

The compositions of the various embodiments described herein may include one or more penetration enhancers. The penetration enhancer in the compositions of various embodiments described above may be present in an amount about 0.5% (w/w) to about 40% (w/w), about 1% (w/w) to about 20% (w/w), about 5% (w/w) to about 15% (w/w) based on the total composition or any range or individual concentration encompassed by these example ranges.

In various embodiments, the aqueous phase may be about 20% (v/v) to about 90% (v/v), about 25% (v/v) to about 80% (v/v), about 30% (v/v) to about 75% (v/v) based on the total volume of the composition, or any range or individual concentration encompassed by these example ranges. The remainder of the composition may be the oil phase or a combination of oil phases.

Other embodiments of the invention include methods for treating hair growth disorders or hair loss by administering the compositions described above. Such methods are not limited to particular indications; however, the compositions described herein can be particularly useful for treating alopecia areata, alopecia totalis, alopecia universalis, vitiligo, and graft versus host disease. Other indications that can be treated by administering the compositions of various embodiments, include telogen effluvium, tinca capitis (dermatophytosis), hypotrichosis, hereditary hypotrichosis simplex, frontal fibrosing alopecia, cicatricial alopecia, lichen planopilaris, ring alopecia, chemotherapy induced alopecia, and the like.

The methods of various embodiments may include the steps of administering compositions described above to an area of skin on a subject in need of treatment. In some embodiments, the step of administering can be carried out one or two times per week, one, two, or three times per month, one to four times per year, and the like and any time period encompassed by these examples. In certain embodiments, administering can be carried out the prescribed number of times per day for one week to six months.

The step of administering can be carried out by various means. For example, administering can be accomplished by injecting the composition to the subdermal and subcutaneous areas of the scalp in need of treatment. In some embodiments, administering may include applying mechanical force or energy to the skin of the subject to facilitate delivery. For example, administering includes injecting the composition into the skin of the subject using microneedles. In further embodiments, administering can be carried out using a tattoo machine or other machines for subcutaneously injecting substances into a subject. As is known in the art, certain means for administering may require the use of particular components of the formulation. Such components are described above and can be appropriately incorporated into the compositions.

As used herein, “including”, “containing” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps and others that are unspecified, provided that the latter “do not materially affect the basic and novel characteristic(s)” of what is being described.

### EXAMPLES

The compositions and methods are further supported by the information provided in the following Examples. It is to be understood that the embodiments described in the Examples are merely illustrative and are not intended to limit the scope of the present disclosure, which will be limited only by the appended claims.

### Example 1

Hair loss is a particularly difficult and sensitive problem for patients suffering from it. Different types of hair loss have different underlying mechanisms. One cause of hair loss is inflammation, which in many of such cases becomes chronic. The inflammation damages the hair follicles, resulting in irreversible alopecia or inflammation-based hair loss. Many of the current products for treating hair loss cause irritation and/or are oily and thick, such that patients do not like using them for cosmetic reasons and in some cases cannot because of the side effects as described in more detail below. New and improved treatments are needed. The liquid compositions, which may also be referred to as solutions or formulations, described herein provide improved treatment options for patients suffering from hair loss, such as that associated with inflammation.

Scarring alopecia is an example of hair loss caused by inflammation that affects more women than men. For reasons that are still unknown, the prevalence of scarring alopecia among the general population in the US is increasing. Scarring alopecia is a particularly debilitating form of alopecia. It is an auto-inflammatory disorder of the scalp that is associated with progressive and permanent (irreversible) hair loss and scarring. The most common forms of scarring alopecia include frontal fibrosing alopecia (FFA), lichen planopilaris (LPP), and central centrifugal cicatricial alopecia (CCCA). Therapeutic options for scarring alopecia in any form are extremely limited, and a cure does not exist to date.

Early diagnosis and treatment is of key importance to halt the progression of this inflammatory disorder. If scarring alopecia is not treated early and successfully, scalp areas of increasing size will be affected by chronic inflammation, followed by the irreversible loss of the hair follicles in these areas. Irreversible loss of hair follicles means that these scalp areas will never be able to grow hair again, and the bald areas are permanent. Given the auto-immune character of scarring alopecia, its association with itching, pain and burning, as well as the progressive, ultimately extensive, irreversible hair loss leading to a substantially reduced quality of life and negative psychological consequences in affected patients, the effective medical treatment of scarring alopecia is mandatory.

As per the official treatment guidelines for scarring alopecia, first-line therapy includes the use of topical immunosuppressants. While topical steroids are available, they must not be used long term. Topical steroids are associated with many side effects typically seen in patients who use steroids long-term, such as thinning of the skin, permanent stretch marks, telangiectasias, and skin discoloration. However, scarring alopecias are chronic disorders, and most patients will require treatment for many years. Topical tacrolimus is a great alternative and superior to topical steroids as topical tacrolimus is not associated with many of the side effects typically seen in patients who use steroids long-term, such as thinning of the skin, permanent stretch marks, telangiectasias, and skin discoloration. Unfortunately, while tacrolimus ointment is commercially available, an ointment cannot be applied to the scalp when hair is still present. Ointments are very thick and greasy, will get caught in patients' hair and not reach the scalp and thus not be effective. Moreover, the ointment will stick to the hair and weigh it down, which will make the hair look even less voluminous. This is extremely problematic for patients as they already fight the consequences of hair loss and have less hair due to their disorder. In contrast, a topical liquid tacrolimus solution, such as those disclosed herein, can be applied easily directly to the scalp and will not get stuck in hair that is present, ensuring that the prescribed medication is effective. Moreover, the non-greasy character and the use of volatile components in the medication will minimize the residue in patients' hair, which will ensure that the hair retains its volume and does not get weighed down.

Importantly, the topical liquid formulations are made without propylene glycol. Although widely used in topical products for skin disorders, even in commercial Rogaine® treatment for hair loss for example, propylene glycol is known to not only cause allergies, but also to lead to scalp redness, scalp itchiness, scalp flaking and scalp discomfort, even in patients who are not directly allergic to propylene glycol. These effects are observed particularly when patients use topicals with propylene glycol for weeks and longer. Since patients with scarring alopecias require chronic treatment, the likelihood is high that these patients will also experience side effects from the chronic use of propylene glycol, which may further exacerbate the manifestations associated with their scarring alopecia. Propylene glycol, however, is often used because its immediate feel is cosmetically elegant, and it is a great drug carrier and/or solvent. To completely avoid any of the side effects associated with the chronic use of topicals that contain propylene glycol, the formulas described here were developed with an alternative named 1,3-propanediol. Even though both liquids are closely related (propylene glycol is also named 1,2-propylene glycol) and share many characteristics, 1,3-propanediol is tolerated much better on the scalp, which is tolerated much better by patients with scarring alopecias in particular as well.

A liquid composition for treating hair loss by topical administration is preferred by patients over compositions with oil or ointments. The liquid compositions may be administered by any means, such as by dropper or spray. The liquid compositions are applied where needed, such as on the scalp, as prescribed to be therapeutically effective, for example at least once weekly up to multiple times per day.

Liquid compositions for topical administration for treating hair loss, for example but not limited to hair loss associated with cicatricial alopecia, frontal fibrosing alopecia, and/or lichen planopilaris, were developed having tacrolimus as the active agent, a solvent such as ethyl alcohol, DMSO, or benzyl alcohol, and a carrier, such as 1,3-propanediol. The compositions include about 0.1% to about 0.5% (w/w) of tacrolimus, about 20% to about 80% (w/w) of at least one solvent, and the remaining, e.g. about 20% to about 80% (w/w), of a carrier based on the total weight of the composition. Example liquid compositions, or solutions, developed for topical administration are shown in Table 1.

TABLE 1

| | Composition 1 | | Composition 2 | | Composition 3 | |
|---|---|---|---|---|---|---|
| Tacrolimus | 0.3 | g | 0.2 | g | 0.1 | g |
| Ethyl alcohol | 65 | ml | 70 | ml | 65 | ml |
| 1,3-Propanediol | 34.7 | ml | 29.8 | ml | 34.9 | ml |

A liquid composition or solution with the active agent tofacitinib was also developed. This formulation has about 1.5% to 4% (w/w) tofacitinib citrate, about 35% (w/w) DMSO and about 36% (w/w) ethyl alcohol as solvents, and the remaining, e.g. about 25% (w/w), 1,3-propaendiol as a carrier based on the total weight of the composition.

### Example 2

In some particularly difficult to treat cases of hair loss, such as those noted above, a combination of active agents may be needed. A topical liquid composition was developed for these cases that include at least one active agent of minoxidil, tofacitinib, tacrolimus, and a steroid, at least one solvent, and a carrier. One topical liquid composition developed herein includes: about 5% (w/w) minoxidil, about 1.5% to 4% (w/w) tofacitinib, 0.1 to about 0.3% tacrolimus, and a steroid, such as about 0.05% (w/w) clobetasol propionate, as the active agents, with ethyl alcohol, DMSO, and lactic acid as solvents, and 1,3-propanediol as the carrier. Another such liquid composition includes: 5 g minoxidil, 3.2 g tofacitinib citrate, 0.1 to 0.3 g tacrolimus, 0.05 g clobetasol propionate, 30.8 to 31 ml 1,3-propanediol, 4.5 ml lactic acid, 21.15 ml ethyl alcohol, and 35 ml DMSO.

Whereas particular features of the present invention have been described above for purposes of illustration, it will be evident to those skilled in the art that numerous variations of the details of the coating composition, coating, and methods disclosed herein may be made without departing from the scope in the appended claims.

What is claimed is:

1. A composition comprising about 0.1% to about 0.5% (w/w) tacrolimus, about 20% to about 80% (w/w) of at least one solvent, and about 20% to about 80% (w/w) of a carrier, wherein the composition is a liquid solution.

2. The composition of claim 1, wherein the at least one solvent is ethyl alcohol, benzyl alcohol, dimethyl sulfoxide (DMSO), lactic acid, or a combination thereof.

3. The composition of claim 2, wherein the solvent is ethyl alcohol.

4. The composition of claim 1, wherein the carrier is 1,3-propanediol.

5. The composition of claim 1, further comprising at least one of about 0.5% to about 10% minoxidil and about 1% to about 5% tofacitinib.

6. The composition of claim 1, further comprising a steroid.

7. The composition of claim 6, wherein the steroid is clobetasol propionate.

8. The composition of claim 6, wherein the clobetasol propionate is about 0.025% to about 1% (w/w) of the total weight of the composition.

9. The composition of claim 5, further comprising lactic acid.

10. The composition of claim 5, further comprising DMSO.

11. The composition of claim 8, wherein the composition comprises:
about 0.1 to 0.3% (w/w) tacrolimus,
about 2% (w/w) tofacitinib,
about 5% (w/w) minoxidil,
about 0.05% (w/w) clobetasol propionate,
about 21% ethyl alcohol, about 4 to about 5% (w/w) lactic acid, and about 35% (w/w)
DMSO as the combination of at least one solvent, and
about 30% to about 32% 1,3-propanediol as the carrier.

12. A method for treating hair loss comprising topically administering the liquid composition of claim 1 to a subject in need thereof.

13. The method of claim 12, wherein the hair loss is associated with inflammation.

14. The method of claim 12, wherein the hair loss is associated with scarring alopecia.

15. The method of claim 12, wherein the hair loss is associated with at least one of cicatricial alopecia, frontal fibrosing alopecia, and lichen planopilaris.

16. The method of claim 12, wherein the liquid composition is administered by dropper or spray.
